(19) United States
(12) Patent Application Publication
(10) Pub. No.: US 2010/0160266 A1
(43) Pub. Date: Jun. 24, 2010

(54) ORGANIC COMPOUNDS

(75) Inventor: Gerd Ascher, Kundl (AT)

Correspondence Address: Workman Nydegger;1000 Eagle Gate Tower, 60 East South Temple, Salt Lake City, UT, 84111, US

(73) Assignee: NABRIVA THERAPEUTICS FORSCHUNGS GMBH, Wien (AT)

(21) Appl. No.: 11/993,813

(22) PCT Filed: Jun. 14, 2006
(86) PCT No.: PCT/AT06/00242

§ 371 Date: Apr. 18, 2008

(30) Foreign Application Priority Data
Jun. 27, 2005 (GB) 0513058.8

(51) Int. Cl. A61K 31/603; C07C 69/74; C07D 211/06; A61K 31/24; A61K 31/4458; A61P 17/10

(52) U.S. Cl. 514/161; 560/117; 546/204; 514/541; 514/325

(57) ABSTRACT

A pleuromutilin in the form of a salt with salicylic acid, azelaic acid, sebacic acid or 2-[(2,6-dichlorophenyl)amino]benzeneacetic acid.

The present invention relates to organic compounds, e.g. mutilins, such as pleuromutilins, which are useful as antimicrobials, e.g. antibiotics.

We have now found salts of pleuromutilins which exhibit surprising activity.

In one aspect the present invention provides a pleuromutilin in the form of a salt with salicylic acid, azelaic acid, sebacic acid or 2-[(2,6-dichlorophenyl)amino]benzeneacetic acid, preferably salicylic acid.

“Pleuromutilin” as used herein are pleuromutilins which have the ability to form a salt with an acid, such as an acid addition salt, e.g. pleuromutilins which comprise functional chemical groups which have the ability to form an acid addition salt upon addition of an acid.

2-[(2,6-dichlorophenyl)amino]benzeneacetic acid is also known as diclofenac.

In another aspect the present invention provides a pleuromutilin, which has the ability to form an acid addition salt, in the form of a salt with salicylic acid, azelaic acid, sebacic acid or 2-[(2,6-dichlorophenyl)amino]benzeneacetic acid, preferably salicylic acid.

Pleuromutilins in the form of a salt with salicylic acid, azelaic acid, sebacic acid or 2-[(2,6-dichlorophenyl)amino]benzeneacetic acid are herein also designated as “compound(s) of (according to) the present invention”.

A pleuromutilin in a compound of the present invention may exist in the form of isomers and mixtures thereof, e.g. including diastereoisomers and mixtures thereof. Isomeric mixtures may be separated as appropriate, e.g. according to a method as conventional, to obtain pure isomers. The present invention includes a pleuromutilin in a compound of the present invention in any isomeric form and in any isomeric mixture, such as described in patent literature cited below, which patent literature is introduced herein by reference with respect to isomeric forms of pleuromutilins. Preferably the configuration in the mutilin ring is the same as in a naturally produced mutilin.

Pleuromutilin, e.g. a compound of formula

Error! Objects cannot be created from editing field codes.

is a naturally occurring antibiotic, e.g. produced by the basidomycetes Pleurotus mutilus and P. passeckerianus, see e.g. The Merck Index, 12th edition, item 7694. A number of further pleuromutilins having the principle ring structure of pleuromutilin and having e.g. antibacterial activity are known.

A pleuromutilin in a compound for the present invention includes a pleuromutilin having the basic structural elements as set out in formula

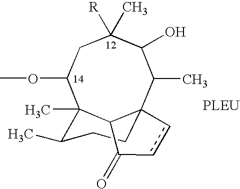

The following numbering system is used in the present application:

Error! Objects cannot be created from editing field codes.

The dotted line between positions 19 an 20 (and between positions 1 and 2) is a bond or is no bond. In a compound of formula A or of formula PLEU a hydrogen atom in positions 4, 7 and/or 8 of the ring system may be replaced by deuterium, and if the dotted line between positions 1 and 2 is no bond (single bond between positions 1 and 2) the ring system may be further substituted in positions 1 and/or 2, e.g. by halogen, deuterium or hydroxy. The group —O— in position 14 is further substituted, preferably by a substituted carbonyl group.

Examples of pleuromutilins in a compound of the present invention include e.g. a compound as disclosed, such as a compound as claimed, in
GB1312148, GB1410505, DE3405632, U.S. Pat. No. 3,987,194, U.S. Pat. No. 4,278,674, U.S. Pat. No. 4,130,709, EP0013768, EP0153277 (e.g. including Valnemulin (Econor®)), US516526, WO9322288, WO9725309, WO9805659, WO9821855, WO9921855, WO0007974, WO0027790, WO0037074, WO0073287, WO0007974, WO0114310, WO0109095, WO0114310, WO0174788, WO0204414, WO0212199, WO0222580, WO0230929, WO0238528, WO0037074, WO2004011431.

Preferred pleuromutilins in a compound of the present invention include e.g. compounds as disclosed in WO0109095, e.g. a compound of formula

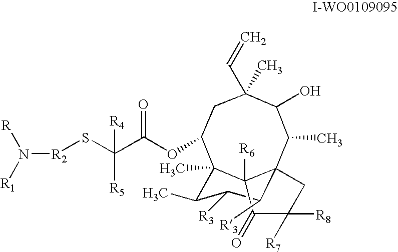

wherein R is hydrogen or alkyl; R₁ is hydrogen or a group of formula

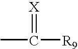

wherein X is S, O, or NR₁₀, wherein R₁₀ is H or alkyl, or N⁺(R′₁₀)₂ wherein R′₁₀ is alkyl in the presence of an appropriate anion; and R₉ is amino, alkyl, aryl, heterocyclyl or mercapto; and, if X is oxygen, R₉ is additionally hydrogen; R₂ is arylene, e.g. phenylene; or heterocyclene; R₄ is hydrogen or alkyl; R₅ is hydrogen or alkyl; R₃, R₃′, R₆, R₇ and R₈ independently of each other are hydrogen or deuterium; or R and R₂ together with the nitrogen atom to which they are attached form non-aromatic heterocyclene and R₁ is a group of formula

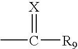

wherein X and R₉ are as defined above; e.g. a compound of formula

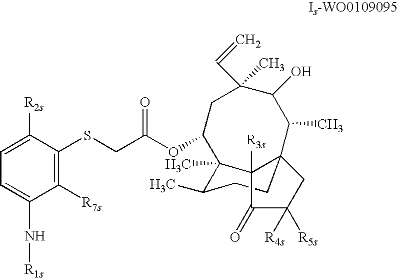

wherein R₁ₛ is hydrogen or a group of formula

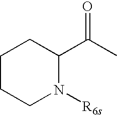

wherein R₆ₛ is hydrogen or deuterium; R₂ₛ is hydrogen, methyl or tert-butyl; R₇ₛ is hydrogen or methyl; and R₃ₛ, R₄ₛ and R₅ₛ are hydrogen or deuterium,
such as a compound of formula

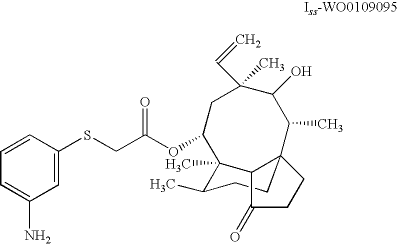

or such as a compound of formula

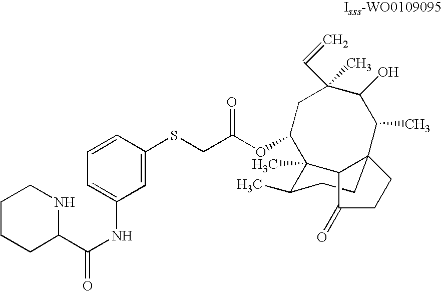

e.g. including a compound of formula

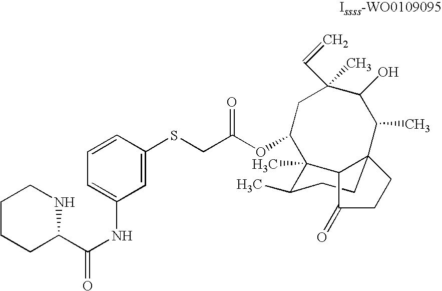

Other preferred pleuromutilins in a compound of the present invention include e.g. compounds as disclosed in WO0204414, e.g. a compound selected from 14-O-[(cycloalkyl-sulfanyl)acetyl]mutilins; 14-O-[(cycloalkyl-alkyl-sulfanyl)acetyl]mutilins; 14-O-[(cycloalkoxy)acetyl]mutilins; or 14-O-[(cycloalkyl-alkoxy)acetyl]mutilins, such as of formula

Error! Objects cannot be created from editing field codes.
wherein R is hydrogen; R₁ is hydrogen or a group of formula
   Error! Objects cannot be created from editing field codes. wherein X is sulphur, oxygen or NR₁₀, wherein R₁₀ is hydrogen or alkyl; and R₉ is amino, alkyl, aryl or heterocyclyl; and, if X is oxygen, R₉ is additionally hydrogen; Y is sulphur or oxygen; R₂ is hydrogen or one or more substituents, R₄ is hydrogen or alkyl; R₅ is hydrogen or alkyl; R₃ and R₃′ are hydrogen, deuterium, or halogen; R₆, R₇ and R₈ are hydrogen or deuterium; m is a number selected from 0 to 4; n is a number selected from 0 to 10; and p is a number selected from 0 to 10; with the proviso that n plus p are at least 1; e.g. a compound of formula

Error! Objects cannot be created from editing field codes.

wherein R₁ₚ is hydrogen or the residue of an amino acid; e.g. a compound of formula

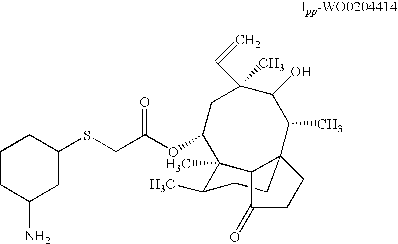

e.g. including compounds of formula

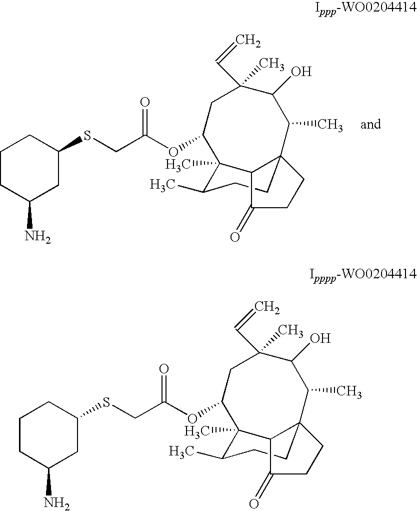

Other preferred pleuromutilins in a compound of the present invention include e.g. a compound as disclosed in WO0222580, of formula

Error! Objects cannot be created from editing field codes.

wherein R and R₂ together with the nitrogen atom to which they are attached form pyrrolidinyl or piperidinyl, R₁ is a group of formula

Error! Objects cannot be created from editing field codes. R₃ and R′₃ are hydrogen, deuterium or halogen, R₄ is hydrogen or alkyl, R₅ is hydrogen or alkyl, R₆, R₇ and R₈ are hydrogen or deuterium; R₉ is amino, alkyl, aryl, heterocyclyl or mercapto; and, if X is oxygen, R₉ is additionally hydrogen; R₁₀ is hydrogen or alkyl, R′₁₀ is alkyl, X is sulphur, oxygen, NR₁₀, or N⁺(R′₁₀)₂ in the presence of an appropriate anion, Y is sulphur or oxygen, and m is 0, 1 or 2;

with the proviso that, when R and R₂ together with the nitrogen atom to which they are attached form piperidinyl, m is 0, Y is S and Y is attached in position 3 of said piperidine ring, that group of formula I which is attached to the piperidine ring via the residue Y is either in the (S)-configuration or in the (R)-configuration, preferably in the (S)-configuration; preferably a compound of formula

Error! Objects cannot be created from editing field codes.

wherein R₃ₚ, R′₃ₚ, R₆ₚ, R₇ₚ and R₈ₚ are, index-number correspondingly, as defined for a compound of formula I-WO0222580 for R₃, R′₃, R₆, R₇ and R₈; and R₅ₚ is hydrogen or one or more substituents, and if the group attached to the piperidine ring via the sulphur atom is in position 3 of said piperidine ring and R₅ₚ is hydrogen, then the group attached to the sulphur atom is either in the (S)-configuration or in the (R)-configuration, such as a compound of formula

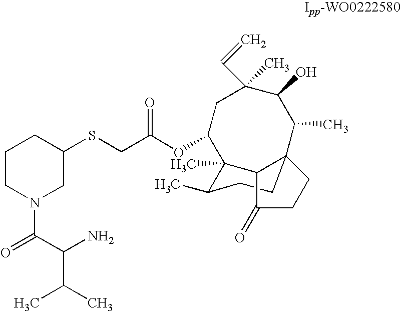

e.g. including compounds of formula

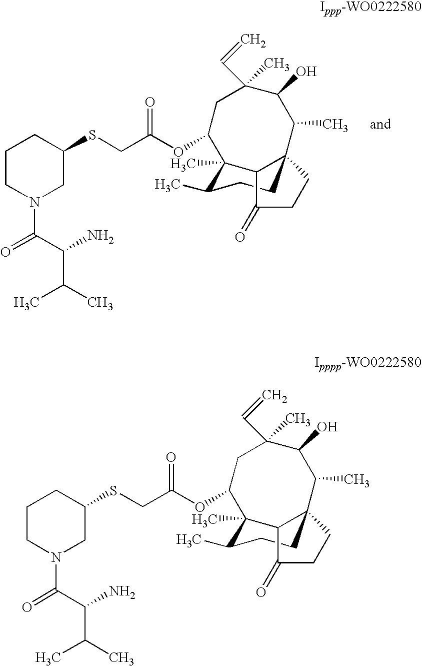

e.g. wherein the group attached to the pleuromutilin via the sulphur atom is either in the S-configuration or in the R-configuration.

In a pleuromutilin in a compound of the present invention each single defined substituent may be a preferred substituent, e.g. independently of each other substituent defined.

In another aspect the present invention provides a compound of formula Iₚₚ-WO0204414, such as a compound of formula Iₚₚₚ-WO0204414 and Iₚₚₚₚ-WO0204414, e.g. wherein the group attached to the pleuromutilin via the sulphur atom is either in the S-configuration or in the R-configuration, in the form of a salt with salicylic acid, azelaic acid, sebacic acid or 2-[(2,6-dichlorophenyl)amino]benzeneacetic acid, preferably salicylic acid.

In another aspect the present invention provides a compound of formula Iₛₛ-WO0109095 in the form of a salt with salicylic acid, azelaic acid, sebacic acid or 2-[(2,6-dichlorophenyl)amino]benzeneacetic acid, preferably salicylic acid.

In another aspect the present invention provides a compound of formula Iₛₛₛ-WO0109095, such as a compound of formula Iₛₛₛₛ-WO0109095, e.g. wherein the group attached to the pleuromutilin via the sulphur atom is either in the S-configuration or in the R-configuration, in the form of a salt with salicylic acid, azelaic acid, sebacic acid or 2-[(2,6-dichlorophenyl)amino]benzeneacetic acid, preferably salicylic acid.

In another aspect the present invention provides a compound of formula Iₚₚ-WO0222580, such as a compound of formula Iₚₚₚ-WO0222580 or Iₚₚₚₚ-WO0222580, e.g. wherein the group attached to the pleuromutilin via the sulphur atom is either in the S-configuration or in the R-configuration, in the form of a salt with salicylic acid, azelaic acid, sebacic acid or 2-[(2,6-dichlorophenyl)amino]benzeneacetic acid, preferably salicylic acid.

In another aspect the present invention provides the compound 14-O—[(N—((R)-Valyl)-piperidine-3(S)-yl)-sulfanylacetyl)-mutilin in the form of a salt with salicylic acid, azelaic acid, sebacic acid or 2-[(2,6-dichlorophenyl)amino]benzeneacetic acid, preferably salicylic acid.

A compound of the present invention may be prepared as appropriate, e.g. according, e.g analogously, to a method as conventional; e.g. a pleuromutilin may be dissolved or suspended in appropriate solvent, preferably organic solvent, e.g. including halogenated hydrocarbons, such as CH₂Cl₂, an alcohol, such as methanol, ethanol, an ether, such as dimethylether, diisopropylether or tetrahydrofuran, a ketone, such as acetone, and the suspension or solution obtained may be treated with salicylic acid, azelaic acid, sebacic acid or 2-[(2,6-dichlorophenyl)amino]benzeneacetic acid, e.g. as such, e.g. or dissolved or suspended in a solvent, e.g. in an amount that is at least equivalent to the amount of the pleuromutilin used. From the mixture obtained a compound of the present invention may be isolated as appropriate, e.g.
from the mixture obtained a solid may precipitate, e.g. in crystalline form, which may be isolated by filtration, centrifugation,
from the mixture obtained solvent may be evaporated off and the evaporation residue may be collected,
the mixture obtained may be subjected to lyophilisation.

A compound of the present invention obtained may be purified as appropriate, e.g. according, e.g. analogously, to a method as conventional, e.g. by extraction and/or by chromatography;

e.g. before or after isolation

The compounds of the present invention exhibit pharmacological activity and are therefore useful as pharmaceuticals, such as antimicrobials or antibiotics, in the treatment of tuberculosis, in the treatment of Helicobacter infections. Such activity is e.g. described in correspondingly cited literature.

We have found that compounds of the present invention are particularly useful in the treatment of acne. E.g. in commercially available in vitro acne assays (the determination of the minimal inhibitory concentration (MIC) is performed according to the approved NCCLS guidelines¹⁾ by agar dilution technique on Wilkins-Chalgren Agar—1) National committee for clinical laboratory standards. Methods for Antimicrobial susceptibility testing of anaerobic bacteria. 1993. (NCCLS document M11-A3 3^{rd} edition. Vol. 13, No. 26) the compound 14-O—[(N—((R)-Valyl)-piperidine-3(S)-yl)-sulfanylacetyl)-mutilin in the form of a salt with salicylic acid shows remarkably better activity than the compound 14-O—[(N—((R)-Valyl)-piperidine-3(S)-yl)-sulfanylacetyl)-mutilin in the form of a hydrochloride.

In another aspect the present invention provides a compound of formula
Iₚₚ-WO0204414, such as a compound of formula Iₚₚₚ-WO0204414 or Iₚₚₚₚ-WO0204414, e.g. wherein the group attached to the pleuromutilin via the sulphur atom is either in the S-configuration or in the R-configuration, in the form of a salt with salicylic acid, azelaic acid, sebacic acid or 2-[(2,6-dichlorophenyl)amino]benzeneacetic acid, preferably salicylic acid,
Iₛₛ-WO0109095 in the form of a salt with salicylic acid, azelaic acid, sebacic acid or 2-[(2,6-dichlorophenyl)amino]benzeneacetic acid, preferably salicylic acid,
Iₛₛ-WO0109095, such as a compound of formula Iₛₛₛ-WO0109095, e.g. wherein the group attached to the pleuromutilin via the sulphur atom is either in the S-configuration or in the R-configuration, in the form of a salt with salicylic acid, azelaic acid, sebacic acid or 2-[(2,6-dichlorophenyl)amino]benzeneacetic acid, preferably salicylic acid, or
Iₚₚ-WO0222580, such as a compound of formula Iₚₚₚ-WO0222580 or Iₚₚₚₚ-WO0222580, e.g. wherein the group attached to the pleuromutilin via the sulphur atom is either in the S-configuration or in the R-configuration, in the form of a salt with salicylic acid, azelaic acid, sebacic acid or 2-[(2,6-dichlorophenyl)amino]benzeneacetic acid, preferably salicylic acid,
   such as the compound 14-O—[(N—((R)-Valyl)-piperidine-3(S)-yl)-sulfanylacetyl)-mutilin in the form of a salt with salicylic acid, azelaic acid, sebacic acid or 2-[(2,6-dichlorophenyl)amino]benzeneacetic acid, preferably salicylic acid,
   for use in the preparation of a medicament for the treatment of acne.

In another aspect the present invention provides a method of treating acne comprising administering an effective amount of a compound of formula
Iₚₚ-WO0204414, such as a compound of formula Iₚₚₚ-WO0204414 or Iₚₚₚₚ-WO0204414, e.g. wherein the group attached to the pleuromutilin via the sulphur atom is either in the S-configuration or in the R-configuration, in the form of a salt with salicylic acid, azelaic acid, sebacic acid or 2-[(2,6-dichlorophenyl)amino]benzeneacetic acid, preferably salicylic acid,
Iₛₛ-WO0109095 in the form of a salt with salicylic acid, azelaic acid, sebacic acid or 2-[(2,6-dichlorophenyl)amino]benzeneacetic acid, preferably salicylic acid,
Iₛₛₛ-WO0109095, such as a compound of formula Iₛₛₛ-WO0109095, e.g. wherein the group attached to the pleuromutilin via the sulphur atom is either in the S-configuration or in the R-configuration, in the form of a salt with salicylic acid, azelaic acid, sebacic acid or 2-[(2,6-dichlorophenyl)amino]benzeneacetic acid, preferably salicylic acid, or
Iₚₚ-WO0222580, such as a compound of formula Iₚₚₚ-WO0222580 or Iₚₚₚₚ-WO0222580, e.g. wherein the group attached to the pleuromutilin via the sulphur atom is either in the S-configuration or in the R-configuration, in the form of a salt with salicylic acid, azelaic acid, sebacic acid or 2-[(2,6-dichlorophenyl)amino]benzeneacetic acid, preferably salicylic acid,
   such as the compound 14-O—[(N—((R)-Valyl)-piperidine-3(S)-yl)-sulfanylacetyl)-mutilin in the form of a salt with salicylic acid, azelaic acid, sebacic acid or 2-[(2,6-dichlorophenyl)amino]benzeneacetic acid, preferably salicylic acid,
   to a subject in need of such treatment.

In another aspect the present invention provides a pharmaceutical composition for the treatment of acne comprising a compound of formula
Iₚₚ-WO0204414, such as a compound of formula Iₚₚₚ-WO0204414 or Iₚₚₚₚ-WO0204414, e.g. wherein the group attached to the pleuromutilin via the sulphur atom is either in the S-configuration or in the R-configuration, in the form of a salt with salicylic acid, azelaic acid, sebacic acid or 2-[(2,6-dichlorophenyl)amino]benzeneacetic acid, preferably salicylic acid,
Iₛₛ-WO0109095 in the form of a salt with salicylic acid, azelaic acid, sebacic acid or 2-[(2,6-dichlorophenyl)amino]benzeneacetic acid, preferably salicylic acid,
Iₛₛₛ-WO0109095, such as a compound of formula Iₛₛₛ-WO0109095, e.g. wherein the group attached to the pleuromutilin via the sulphur atom is either in the S-configuration or in the R-configuration, in the form of a salt with salicylic acid, azelaic acid, sebacic acid or 2-[(2,6-dichlorophenyl)amino]benzeneacetic acid, preferably salicylic acid, or
Iₚₚ-WO0222580, such as a compound of formula Iₚₚₚ-WO0222580 or Iₚₚₚₚ-WO0222580, e.g. wherein the group attached to the pleuromutilin via the sulphur atom is either in the S-configuration or in the R-configuration, in the form of a salt with salicylic acid, azelaic acid, sebacic acid or 2-[(2,6-dichlorophenyl)amino]benzeneacetic acid, preferably salicylic acid,
   such as the compound 14-O—[(N—((R)-Valyl)-piperidine-3(S)-yl)-sulfanylacetyl)-mutilin in the form of a salt with salicylic acid, azelaic acid, sebacic acid or 2-[(2,6-dichlorophenyl)amino]benzeneacetic acid, preferably salicylic acid,
   in association with at least one pharmaceutical excipient, e.g. appropriate carrier and/or diluent, e.g. including fillers, binders, disintegrators, flow conditioners, lubricants, sugars and sweeteners, fragrances, preservatives, stabilizers, wetting agents and/or emulsifiers, solubilizers, salts for regulating osmotic pressure and/or buffers.

For pharmaceutical use a compound of the present invention includes one or more, preferably one, compounds of the present invention, e.g. a combination of two or more compounds of the present invention.

Treatment includes treatment and prophylaxis.

For such treatment, the appropriate dosage will, of course, vary depending upon, for example, the chemical nature and the pharmakokinetic data of a compound of the present invention employed, the individual host, the mode of administration and the nature and severity of the conditions being treated. However, in general, for satisfactory results in larger mammals, for example humans, an indicated daily dosage is in the range from about 0.01 g to about 1.0 g of a compound of the present invention; conveniently administered, for example, in divided doses up to four times a day.

A compound of the present invention may be administered by any conventional route, for example enterally, e.g. including nasal, buccal, rectal, oral, administration; parenterally, e.g. including intravenous, intramuscular, subcutaneous administration; or topically; e.g. including epicutaneous, intranasal, intratracheal administration;

e.g. in form of coated or uncoated tablets, capsules, (injectable) solutions, solid solutions, suspensions, dispersions, solid dispersions; e.g. in the form of ampoules, vials, in the form of creams, gels, pastes, inhaler powder, foams, tinctures, lip sticks, drops, sprays, or in the form of suppositories.

A compound of the present invention may be used for pharmaceutical treatment according to the present invention alone, or in combination with one or more other pharmaceutically active agents. Such other pharmaceutically active agents include agents which are appropriate for the treatment of inflammation, e.g. including skin inflammation, including agents useful for the treatment of acne.

In another aspect the present invention provides a pharmaceutical composition according to the present invention further comprising another agent which is appropriate in the treatment of skin inflammation, e.g. acne.

Combinations include fixed combinations, in which two or more pharmaceutically active agents are in the same formulation; kits, in which two or more pharmaceutically active agents in separate formulations are sold in the same package, e.g. with instructions for co-administration; and free combinations in which the pharmaceutically active agents are packaged separately, but instructions for simultaneous or sequential administration are given.

Such compositions may be manufactured according, e.g. analogously to a method as conventional, e.g. by mixing, granulating, coating, dissolving or lyophilizing processes. Unit dosage forms may contain, for example, from about 0.5 mg to about 1000 mg, such as 1 mg to about 500 mg.

In the following Examples all temperatures are in degrees Celsius (° C.) and are uncorrected.

### EXAMPLE 1

### 14-O—[(N—((R)-Valyl)-piperidine-3(S)-yl)-sulfanylacetyl)-mutilin in the Form of a Salt with Salicylic Acid

1.38 g (10 mmol) of salicylic acid are added to a stirred cooled (5°) solution of 5.76 g (10 mmol) of 14-O—[(N—((R)-Valyl)-piperidine-3(S)-yl)-sulfanyl-acetyl)-mutilin in 30 ml of CH₂Cl₂. The mixture obtained is stirred for 15 minutes at room temperature and from the mixture obtained solvent is evaporated to dryness under reduced pressure. Amorphous material obtained is dried for further 5 hours at reduced pressure.

14-O—[(N—((R)-Valyl)-piperidine-3(S)-yl)-sulfanylacetyl)-mutilin in the form of a salt with salicylic acid is obtained.

¹HNMR (DMSO-d₆, ˜1:1 mixture of stable rotamers):

7.7-8.3 (b, 3H, NH₃⁺), 6.6, 7.1, 7.6 (3×m, 4H, salicylate), 6.15 (2×dd,

### 1H, H19, J=17.6 Hz, J=11.2 Hz), 5.6 (2×d, 1H, H14, J=8.2 Hz), 5.05 (m, 2H, H2O),

### 4.5 (b, 1H, 11-OH), 4.08 (dd, 0.5H, H2-Piperidin, J=13.7 Hz, J=3.3 Hz),

3.08 (dd, 0.5H, H2-Piperidin, J=13.7 Hz, J=9.8 Hz), 3.89 (dd, 0.5H, H2-Piperidin, J=13.1 Hz, J=3.2 Hz), 3.41 (m, 0.5H, H2-Piperidin), AB-System: Error! Objects cannot be created from editing field codes._{A}=3.44, Error! Objects cannot be created from editing field codes._{B}=3.33 (2H, SCH₂, J=14.9 Hz), 2.83, 2.96 (2×m, 1H, CHS), 2.4 (b, 1H, H4), 1.34 (s, 3H, CH₃-15), 1.05 (s, 1H, CH₃-18), 0.9, 1.0 (2×m, 6H, (CH₃)₂-Val), 0.81 (d, 3H, CH₃-17, J=6.9 Hz), 0.63 (m, 3H, CH₃-16).

### EXAMPLE 2

### 14-O—[(N—((R)-Valyl)-piperidine-3(S)-yl)-sulfanylacetyl)mutilin in the Form of a Salt with Sebacic Acid

The salt is prepared analogously to Example 1.

¹H-NMR (DMSO-d₆, ˜1:1 mixture of stable rotamers):

6.12 (2×dd, 1H, H-19, J=17.1 Hz, J=11.1 Hz), 5.55 (2×d, 1H, H-14, J=8.6 Hz), 5.05 (m, 2H, H-20), 4.5 (b, 1H, 11-OH), 3.98 (m, 1.5H, H-2 piperidine), 3.49 (m, 1.5H, H-2 piperidine, α-H Val), 3.42 (d, 1H, H-11, J=6 Hz), 3.30 (m, 0.5H, H-2 piperidine), 3.27 (m, 2H, H-22), 3.10 (2×m, 2×0.5H, H-2 piperidine), 2.86 (m, 1.5H, H-2 piperidine, CH—S), 2.4, (b, 1H, H-4), 2.22-2.02 (m, 4H, H-2, 10, 13a), 2.14 (t, 4H, α-H sebacate), 1.92-1.98 (m, 1H, H-4 piperidine), 1.58-1.72 (m, 4H, β-H Val, 1a, 8a, H-3 piperidine), 1.42-1.52 (m, 8H, H-6,7a, H-3 piperidine, H-4 piperidine, β-H sebacate), 1.34 (s, 3H, CH₃-15), 1.20-1.32 (m, 11H, H-1, 7, 13, γ-H sebacate, δ-sebacate), 1.05 (s, 3H, CH₃-18), 1.00 (m, 1H, H-8), 0.87, 0.75 (2×m, 6H, (CH₃)₂-Val), 0.80 (d, 3H, CH₃-17, J=6.8 Hz), 0.62 (m, 3H, CH₃-16).

### EXAMPLE 3

### 14-O—[(N—((R)-Valyl)-piperidine-3(S)-yl)-sulfanylacetyl)mutilin in the Form of a Salt with Azelaic Acid

The salt is prepared analogously to Example 1.

¹H-NMR (DMSO-d₆, ˜1:1 mixture of stable rotamers):

6.12 (2×dd, 1H, H-19, J=17.1 Hz, J=11.1 Hz), 5.55 (2×d, 1H, H-14, J=8.6 Hz), 5.05 (m, 2H, H-20), 4.5 (b, 1H, 11-OH), 3.98 (m, 1.5H, H-2 piperidine), 3.49 (m, 1.5H, H-2 piperidine, α-H Val), 3.42 (d, 1H, H-11, J=6 Hz), 3.30 (m, 0.5H, H-2 piperidine), 3.27 (m, 2H, H-22), 3.10 (2×m, 2×0.5H, H-2 piperidine), 2.86 (m, 1.5H, H-2 piperidine, CH—S), 2.4, (b, 1H, H-4), 2.22-2.02 (m, 4H, H-2, 10, 13a), 2.14 (t, 4H, α-H azelate), 1.92-1.98 (m, 1H, H-4 piperidine), 1.58-1.72 (m, 4H, β-H Val, 1a, 8a, H-3 piperidine), 1.42-1.52 (m, 8H, H-6, 7a, H-3 piperidine, H-4 piperidine, β-H azelate), 1.34 (s, 3H, CH₃-15), 1.20-1.32 (m, 9H, H-1, 7, 13, γ-H azelate, δ-H azelate), 1.05 (s, 3H, CH₃-18), 1.00 (m, 1H, H-8), 0.87, 0.75 (2×m, 6H, (CH₃)₂-Val), 0.80 (d, 3H, CH₃-17, J=6.8 Hz), 0.62 (m, 3H, CH₃-16).

1. A compound comprising: a pleuromutilin in the form of a salt with salicylic acid, azelaic acid, sebacic acid or 2-[(2,6-dichlorophenyl)amino]benzeneacetic acid.

2. A compound comprising: formula 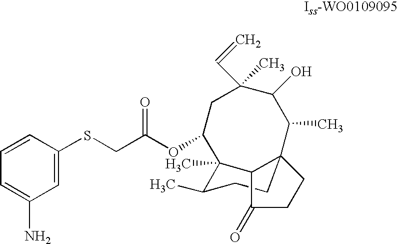
in the form of a salt with salicylic acid, azelaic acid, sebacic acid or 2-[(2,6-dichlorophenyl)amino]benzeneacetic acid.

3. A compound comprising: formula 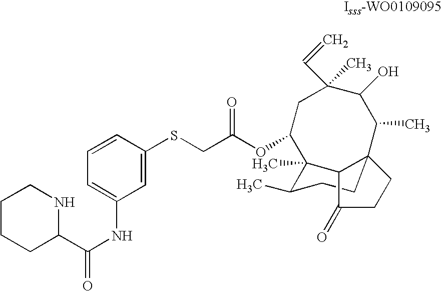
in the form of a salt with salicylic acid, azelaic acid, sebacic acid or 2-[(2,6-dichlorophenyl)amino]benzeneacetic acid.

4. A compound comprising: formula 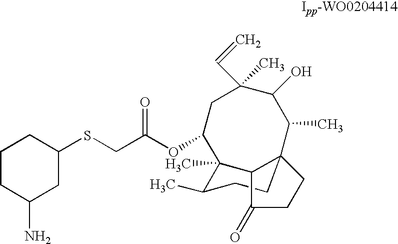
in the form of a salt with salicylic acid, azelaic acid, sebacic acid or 2-[(2,6-dichlorophenyl)amino]benzeneacetic acid.

5. A compound comprising: formula 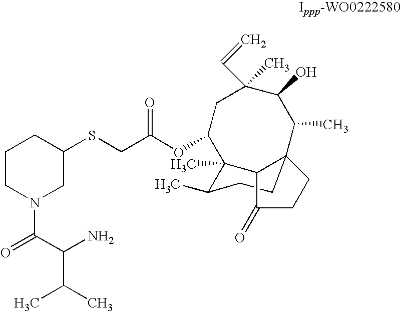
in the form of a salt with salicylic acid, azelaic acid, sebacic acid or 2-[(2,6-dichlorophenyl)amino]benzeneacetic acid.

6. A compound comprising: 14-O—[(N—((R)-Valyl)-piperidine-3(S)-yl)-sulfanylacetyl)-mutilin in the form of a salt with salicylic acid, azelaic acid, sebacic acid or 2-[(2,6-dichlorophenyl)amino]benzeneacetic acid.

7. A compound according to any preceding claim in the form of a salt with salicylic acid.

8. A composition comprising: a compound as defined in any one of claims 2 to 6 in an amount for use in the preparation of a medicament for the treatment of acne.

9. A method of treating acne comprising administering an effective amount of a compound as defined in any one of claims 2 to 6 to a subject in need of such treatment.

10. A pharmaceutical composition for the treatment of acne, comprising a compound as defined in any one of claims 2 to 6 in association with at least one pharmaceutical excipient.
